(19) United States
(12) Patent Application Publication
(10) Pub. No.: US 2003/0236214 A1
(43) Pub. Date: Dec. 25, 2003

(54) Charge reversal of polyion complexes and treatment of peripheral occlusive disease

(76) Inventors: Jon A. Wolff, Madison, WI (US); Vladimir G. Budker, Middleton, WI (US); Julia Hegge, Monona, WI (US); James E. Hagstrom, Middleton, WI (US); Vladimir Trubetskoy, Madison, WI (US)

Correspondence Address: Mark K. Johnson, Mirus Corporation, 505 S. Rosa Rd., Madison, WI, 53719, US

(21) Appl. No.: 10/413,945

(22) Filed: Apr. 15, 2003

Related U.S. Application Data
(63) Continuation-in-part of application No. 09/328,975, filed on Jun. 9, 1999.

(51) Int. Cl. A61K 48/00

(52) U.S. Cl. 514/44

(57) ABSTRACT

A process is described for the delivery of a therapeutic polynucleotide to a tissue suffering from or potentially suffering from ischemia. An ionic polymer is utilized in “recharging” (another layer having a different charge) a condensed polynucleotide complex for purposes of nucleic acid delivery to a cell. The resulting recharged complex can be formed with an appropriate amount of positive or negative charge such that the resulting complex has the desired net charge.

### CROSS-REFERENCE TO RELATED APPLICATIONS

This application is a Continuation-In-Part of the following: U.S. Ser. No. 09/328,975 filed on Jul. 9, 1999

### FIELD OF THE INVENTION

The invention relates to compounds and methods for use in biologic systems. More particularly, polyions are utilized for reversing the charge (“recharging”) particles, such as molecules, polymers, nucleic acids and genes for delivery to cells.

### BACKGROUND OF THE INVENTION

The invention relates to compounds and methods for use in biologic systems. More particularly, polyions are utilized for modifying the charge (“recharging”) particles, such as molecules, polymers, nucleic acids and genes for delivery to cells.

Background Polymers are used for drug delivery for a variety of therapeutic purposes. Polymers have also been used in research for the delivery of nucleic acids (polynucleotides and oligonucleotides) to cells with an eventual goal of providing therapeutic processes. Such processes have been termed gene therapy or anti-sense therapy. One of the several methods of nucleic acid delivery to the cells is the use of DNA-polycation complexes. It has been shown that cationic proteins like histones and protamines or synthetic polymers like polylysine, polyarginine, polyornithine, DEAE dextran, polybrene, and polyethylenimine may be effective intracellular delivery agents while small polycations like spermine are ineffective. The following are some principles involving the mechanism by which polycations facilitate uptake of DNA:

Polycations provide attachment of DNA to the target cell surface. The polymer forms a cross-bridge between the polyanionic nucleic acids and the polyanionic surfaces of the cells. Polycations protect DNA in complexes against nuclease degradation. Polycations can also facilitate DNA condensation. The volume which one DNA molecule occupies in a complex with polycations is drastically lower than the volume of a free DNA molecule. The size of a DNA/polymer complex is important for gene delivery in vivo.

In terms of intravenous injection, DNA must cross the endothelial barrier and reach the parenchymal cells of interest. The largest endothelia fenestrae (holes in the endothelial barrier) occur in the liver and have an average diameter of 100 nm. The trans-epithelial pores in other organs are much smaller, for example, muscle endothelium can be described as a structure which has a large number of small pores with a radius of 4 nm, and a very low number of large pores with a radius of 20-30 nm. The size of the DNA complexes is also important for the cellular uptake process. After binding to the target cells the DNA-polycation complex should be taken up by endocytosis. Since the endocytic vesicles have a homogenous internal diameter of about 100 nm in hepatocytes and are of similar size in other cell types, DNA complexes smaller than 100 nm are preferred.

Condensation of DNA

A significant number of multivalent cations with widely different molecular structures have been shown to induce condensation of DNA. Two approaches for compacting (used herein as an equivalent to the term condensing) DNA: 1. Multivalent cations with a charge of three or higher have been shown to condense DNA. These include spermidine, spermine, Co(NH3)63+,Fe3+, and natural or synthetic polymers such as histone H1, protamine, polylysine, and polyethylenimine. Analysis has shown DNA condensation to be favored when 90% or more of the charges along the sugar-phosphate backbone are neutralized. 2. Polymers (neutral or anionic) which can increase repulsion between DNA and its surroundings have been shown to compact DNA. Most significantly, spontaneous DNA self-assembly and aggregation process have been shown to result from the confinement of large amounts of DNA, due to excluded volume effect.

Depending upon the concentration of DNA, condensation leads to three main types of structures: 1) In extremely dilute solution (about 1 μg/mL or below), long DNA molecules can undergo a monomolecular collapse and form structures described as toroid. 2) In very dilute solution (about 10 μgs/mL) microaggregates form with short or long molecules and remain in suspension. Toroids, rods and small aggregates can be seen in such solution. 3) In dilute solution (about 1 mg/mL) large aggregates are formed that sediment readily.

Toroids have been considered an attractive form for gene delivery because they have the smallest size. While the size of DNA toroids produced within single preparations has been shown to vary considerably, toroid size is unaffected by the length of DNA being condensed. DNA molecules from 400 bp to genomic length produce toroids similar in size. Therefore one toroid can include from one to several DNA molecules. The kinetics of DNA collapse by polycations that resulted in toroids is very slow. For example DNA condensation by Co(NH3)6C13 needs 2 h at RT.

The mechanism of DNA condensation is not clear. The electrostatic force between unperturbed helices arises primarily from a counterion fluctuation mechanism requiring multivalent cations and plays a major role in DNA condensation. The hydration forces predominate over electrostatic forces when the DNA helices approach closer then a few water diameters. In a case of DNA—polymeric polycation interactions, DNA condensation is a more complicated process than the case of low molecular weight polycations. Different polycationic proteins can generate toroid and rod formation with different size DNA at a ratio of positive to negative charge of 0.4. T4 DNA complexes with polyarginine or histone can form two types of structures; an elongated structure with a long axis length of about 350 nm (like free DNA) and dense spherical particles. Both forms exist simultaneously in the same solution. The reason for the co-existence of the two forms can be explained as an uneven distribution of the polycation chains among the DNA molecules. The uneven distribution generates two thermodynamically favorable conformations.

The electrophoretic mobility of DNA-polycation complexes can change from negative to positive in excess of polycation. It is likely that large polycations don't completely align along DNA but form polymer loops that interact with other DNA molecules. The rapid aggregation and strong intermolecular forces between different DNA molecules may prevent the slow adjustment between helices needed to form tightly packed orderly particles.

Cationic molecules with charge greater than +2 are able to condense DNA into compact structures (Bloomfield V. A., DNA condensation, (1996) Curr, Opion in Struct. Biol., 6:334-341). This phenomenon plays a role in chromatin and viral assembly and is of particular importance in the construction of artificial gene delivery vectors. Morphologies of condensed DNA during titration of DNA with polycations are now well documented. When DNA is in excess (DNA/polycation charge ratio >1), complexes assemble into “daisy-shaped” particles that stabilized with loops of uncondensed DNA (Hansma, G. H., Golan, R., Hsieh, W., Lollo, C. P., Mullen-Ley, P. and Kwoh. D. (1998) DNA condensation for gene therapy as monitored by atomic force microscopy, Nucleic Acids Res. 26:2481-2487). When polycation is in excess (DNA/polycation ratio <1), DNA condenses completely within particles that adopt customarily toroid morphology (Tang, M. X., and Szoka, F. C., Jr. 1997, The influence of polymer structure on the interactions of cationic polymers with DNA and morphology of the resulting complexes, Gene Ther. 4:823-832). In low salt aqueous solutions the excess of polycation stabilizes these highly condensed structures and maintains them in soluble state (Kabanov A V, Kabanov V A., Interpolyelectrolyte and block ionomer complexes for gene delivery: physico-chemical aspects, Adv. Drug Delivery Rev. 30:49-60 (1998)).

Several methods can be used to determine the condensation state of DNA. They include the prevention of fluorescent molecules such as etbidium bromide from intercalating into the DNA. The condensation state of DNA was monitored as previously described (Dash, R R, Toncheva V, Schacht E, Seymour L W J. Controlled Release 48:269-276). Alternatively the condensation of fluorescein-labeled DNA (or any fluorescent group) causes self-quenching by bringing the fluorescent groups on the DNA closer together (Trubetskoy, V S, Budker, V G, Slattum, P M, Hagstrom, J E and Wolff, J A. Analytical Biochemistry 267:309-313, 1999).

As previously stated, preparation of polycation-condensed DNA particles is of particular importance for gene therapy, more specifically, particle delivery such as the design of non-viral gene transfer vectors. Optimal transfection activity in vitro and in vivo can require an excess of polycation molecules. However, the presence of a large excess of polycations may be toxic to cells and tissues. Moreover, the non-specific binding of cationic particles to all cells forestalls cellular targeting. Positive charge also has an adverse influence on biodistribution of the complexes in vivo.

Cationic lipid(CL)/DNA complexes (lipoplexes) can be used as gene delivery vehicles in vitro and in vivo. A number of groups have reported successful delivery and expression of reporter genes upon intravenous injection of DNA/CL complexes. High levels of expression were achieved with N-[1-(2,3-dioleyloxy)propyl-N,N,N-trimethylammonium] chloride (DOTMA) [Song Y K, Liu D, Biochim. Biophys. Acts (1998) 1372, 141-150], 1-[2-(9(Z)-octadecenoyloxy)-ethyl]-2-(8(Z)-heptadecenyl)-3-(2-hydroxyethyl)-imidazolinium chloride (DOTIM) [Liu Y, Mounkes L C, Liggitt H D, Brown C S, Solodin I, Heath T D, Debs R J (1997) Nature Biotech. 15, 167-173] and 1,2-bis(oleoyloxy)-3-(trimethylammonio) propane (DOTAP) [Smyth Tempeleton N, Lasic D D, Frederik P M, Strey H H, Roberts D D, Pavlakis G N Nature Biotech. (1997) 15, 647-652). Lung has been found to be primary site for accumulation and transgene expression. All above listed CL require helper lipids to be included in the preparation to achieve maximum activity in vivo. Cholesterol and Tween 80 can be used as such helper additives. No other additives are required for in vivo activity. It has previously been shown that polyanions (PA) of both artificial and natural origin inhibit CL-mediated gene transfer in vitro and in vivo. For example, Barron et al. (Barron L G, Gagne L, Szoka, Jr. F C (1999) Human Gene Ther. 10, 1683-1694) have used injections of anionic liposomes and dextran sulfate to inhibit gene transfer in lungs, heart and liver after i/v administration of DOTAP/colesterol/DNA complexes. Belting and Petersson (Belting M, Petersson P (1999) J. Biol. Chem. 274, 19375-19382) have demonstrated that secreted negatively charged proteoglycans effectively inhibit in vitro CL-mediated transfection of cultured cells. Xu and Szoka, Jr. have demonstrated that polyanions with high charge density disrupt DNA/CL complexes and release free DNA (Xu Y, Szoka, Jr. FC (1996) Biochemistry 35, 5616-5623).

Target Tissues:

The liver is a relatively large organ and is the secretory source of a large amount of serum proteins. The liver sinusoidal endothelial fenestrae are ˜150 nm in diameter which essentially allows parenchymal hepatocytes to come in direct contact blood plasma. These physical and functional characteristics are major factors that rendered liver as an important target tissue for gene therapy. However, in order for gene delivery vectors to take advantage of the “leaky” sinusoids to reach liver hepatocytes, target cells for gene expression, via the vasculature, they must possess certain physical properties: a) stability in physiological salt solutions and serum components; b) optimal vector size which is comparable to sinusoidal fenestrae; c) the ability to interact will cell membrane and induce internalization mechanisms.

### SUMMARY

In order to avoid unwanted effects, anionic particles containing an excess of DNA and cell receptor ligands for targeting have been developed. The present invention describes a process for negatively charging DNA particles by recharging fully condensed polycation/DNA complexes with polyions.

In a preferred embodiment, a process is described for delivering a complex to a cell, comprising, forming a compound having a net charge comprising a polyion and a polymer in a solution, adding a charged polymer to the solution in sufficient amount to form the complex having a net charge different from the compound net charge; and, inserting the complex into a mammal.

In another preferred embodiment, a complex for delivering a polyion to a cell, is described, comprising a polyion and a charged polymer wherein the polyion and the charged polymer are bound in complex, the complex having a net charge that is the same as the net charge of the charged polymer.

In another preferred embodiment a drug for delivery to a cell, is described, comprising a polycation non-covalently attached to a polyanion complexed with a negatively charged polyion.

In another preferred embodiment, DNA/polycation (PC) complexes recharged with various polyanions (PA) can be used for gene delivery in vitro and in vivo. Precise titration of DNA/PC complex with PA results in a significant increase in gene transfer activity both in vitro and in vivo in a narrow range of PA concentrations. Our method involves the use of PA with high charge density and DNA/CL composition possessing in vivo gene transfer activity. The essence of this embodiment is that PA added to DNA/CL results in increased gene transfer activity.

In yet another embodiment, generating small particles that are stable in physiologic salt and serum by to condensing DNA using a polycation and then reversing the net charge of the complexes with the addition of a polyanion. The polycation and polyanion can be stabilized by using a cross-linking reagent.

The examples demonstrate that negatively charged DNA containing particles are stable in salt and serum, with sizes <150 nm. The examples also indicate that negatively charged complexes that are stable in physiological solutions, whether containing DNA or other therapeutic agents, can be targeted to cells in vivo.

In a preferred embodiment, the process may be used to deliver a therapeutic polynucleotide to a muscle cell for the treatment of vascular disease or occlusion. The delivered polynucleotide can express a protein or peptide that stimulates angiogenesis, vasculogenesis, arteriogenesis, or anastomoses to improve blood flow to a tissue. The gene may be selected from the list comprising: VEGF, VEGF II, VEGF-B, VEGF-C, VEGF-D, VEGF-E, VEGF₁₂₁, VEGF₁₃₈, VEGF₁₄₅, VEGF₁₆₅, VEGF₁₈₉, VEGF₂₀₆, hypoxia inducible factor 1α (HIF 1α), endothelial NO synthase (eNOS), iNOS, VEFGR-1 (Flt1), VEGFR-2 (KDR/Flk1), VEGFR-3 (Flt4), neuropilin-1, ICAM-1, factors (chemokines and cytokines) that stimulate smooth muscle cell, monocyte, or leukocyte migration, anti-apoptotic peptides and proteins, fibroblast growth factors (FGF), FGF-1, FGF-1b, FGF-1c, FGF-2, FGF-2b, FGF-2c, FGF-3, FGF-3b, FGF-3c, FGF-4, FGF-5, FGF-7, FGF-9, acidic FGF, basic FGF, hepatocyte growth factor (HGF), angiopoietin 1 (Ang-1), angiopoietin 2 (Ang-2), Platelet derived growth factors (PDFGs), PDGF-BB, monocyte chemotactic protein-1, granulocyte macrophage-colony stimulating factor, insulin-like growth factor-1 (IGF-1), IGF-2, early growth response factor-1 (EGR-1), ETS-1, human tissue kallikrein (HK), matrix metalloproteinase, chymase, urokinase-type plasminogen activator and heparinase. The protein or peptide may be secreted or stay within the cell. For proteins and peptides that are secreted, the gene may contain a sequence that codes for a signal peptide. The delivered polynucleotide can also suppress or inhibit expression of an endogeneous gene or gene product that inhibits angiogenesis, vasculogenesis, arteriogenesis or anastomosis formation. Multiple polynucleotides or polynucleotides containing more that one therapeutic gene may be delivered using the described process. The gene or genes can be delivered to stimulate vessel development, stimulate collateral vessel development, promote peripheral vascular development, improve blood flow in a muscle tissue, or to improve abnormal cardiac function. The gene or genes can also be delivered to treat peripheral circulatory disorders, myocardial disease, myocardial ischemia, limb ischemia, arterial occlusive disease, peripheral arterial occlusive disease, vascular insufficiency, vasculopathy, arteriosclerosis obliterans, thromboangiitis obliterans, atherosclerosis, aortitis syndrome, Behcet's disease, collagenosis, ischemia associated with diabetes, claudication, intermittent claudication, Raynaud disease, cardiomyopathy or cardiac hypertrophy. The polynucleotide can be delivered to a muscle cell that is suffering from ischemia or a normal muscle cell. The muscle cell can be a cardiac cell or a skeletal muscle cell. A preferred skeletal muscle cell is a limb skeletal muscle cell. The polynucleotides can also be delivered to a cells in a tissue that is at risk of suffering from ischemia or a vascular disease or disorder.

Reference is now made in detail to the preferred embodiments of the invention, examples of which are illustrated in the accompanying drawings.

### BRIEF DESCRIPTION OF THE DRAWINGS

FIGS. 1A-1C. Illustration of F1-DNA decondensation during titration of F1-DNA/PLL complex (1:3 charge ratio, F1-DNA=20 μg/ml, 25 mM HEPES, pH 7.5) with different polyanions. (B) Titration of DNA/PLL (1:3 charge ratio, DNA=20 μg/ml, 25 mM HEPES, pH 7.5) complex with SPLL as assessed by light scattering methods. Intensity of scattered light (190) was measured using spectrofluorimeter. Percentage of particles <100 nm in diameter was measured using particle size analyzer as described in the specification. COOH/NH₂ ratios were calculated on the basis of mol weights of N-succinyl lysine and lysine monomers in SPLL and PLL respectively. (C) Zeta potential changes during titration of DNA/PLL complex (1:3 charge ratio, DNA=20 micrograms/ml, 25 mM HEPES, pH 7.5) with SPLL.

FIG. 2 Illustration of atomic force microscopy images of DNA/PLL/SPLL complexes (1:3:10 initial ratio) absorbed on mica in 25 mM HEPES, pH 7.5 as described in the specification.

FIGS. 3A-3B. (A) Illustration of visible spectra of DNA complexes isolated after Rh-DNA/F1-PLL/SPLL (1:3:10) ultracentrifugation and Rh-DNA/F1-PLL (1:1) standard dissolved in 2.5 M NaCl. (B) Visible spectra of DNA complexes isolated after Rh-DNA/PLL/F1-SPLL (1:3:10) ultracentrifugation and Rh-DNA/F1-SPLL (1:1) standard in the same conditions.

FIG. 4. Illustration of transfection efficacy of DNA/PEI complexes recharged with increasing amounts of SPLL polyanion. DNA/PEI/SPLL complexes (2 micrograms DNA, 4 micrograms PEI) were added to HUH7 cells in bovine serum. After 4 hrs of incubation serum with DNA was replaced with fresh OPTI-MEM culture medium with 10% fetal serum. Cells were harvested for luciferase assay 48 hrs after transfection.

FIG. 5. Graph shows in vitro transgene activity of DNA/Lipofectamine complexes recharged with polyglutamic acid.

FIG. 6. Graph of in vitro transgene activity of DNA/LT1 complexes recharged with polymethacrylic acid and dextran sulfate.

FIG. 7. Graph showing transgene activity in lung after i/v administration of DNA/DOTAP:cholesterol/PAA complexes.

FIG. 8. Scan of mouse hepatocytes showing delivery of cross-linked Cy3-DNA/PLL/SPLL particles by tail vein injection. H indicates hepatocytes, S indicates sinusoidal Kupffer and endothelial cells.

FIG. 9. Scan of mouse hepatocytes showing delivery of cross-linked Cy3-DNA/pAllylamine-cys/pAA-thioester complexes by tail vein injection. H indicates hepatocytes, S indicates sinusoidal cells.

FIG. 10. Paraffin cross sections of the Pronator quadratus muscles stained with hematoxylin and eosin and examined under light microscope. Left panel—Pronator quadratus muscle transfected with VEGF-165 plasmid. Right panel—Pronator quadratus muscle transfected with EPO plasmid. Top left picture (VEGF-165) demonstrates increased number of vessels and interstitial cells (presumably—endothelial cells), as compared to right picture (EPO-control), magnification ×200. Bottom left picture (VEGF-165) demonstrates increased number of vessels, most small arteries and capillaries, as compare to right picture (EPO-control). Arrows indicate obvious vascular structures, magnification ×6300.

FIG. 11. Paraffin cross sections of the Pronator quadratus muscles immunostained for endothelial cell marker—CD31, and examined under confocal laser scanning microscope LSM 510, magnification ×400. CD31 marker visualized with Cy3 (black), nuclei with nucleic acid stains To Pro-3. Muscle fibers and red blood cells were visualized by 488 nm laser having autofluorescent emission. Left picture—Pronator quadratus muscle transfected with VEGF-165 plasmid, demonstrates increased of endothelial cells and small vessels, as compare to right picture (EPO-control). The number of CD31 positive cells was increased significantly in VEGF-165 transfected muscle by 61.7% (p<0.001).

### DETAILED DESCRIPTION

Abbreviations: Poly-L-Lysine (PLL), succinic anhydride-PLL (SPLL), polymethacrylic acid, pMAA and polyaspartic acid, pAsp

Gene therapy research may involve the biological pH gradient that is active within organisms as a factor in delivering a polynucleotide to a cell. Different pathways that may be affected by the pH gradient include cellular transport mechanisms, endosomal disruption/breakdown, and particle disassembly (release of the DNA).

Gradients that can be useful in gene therapy research involve ionic gradients that are related to cells. For example, both Na⁺ and K⁺ have large concentration gradients that exist across the cell membrane. Recharging systems can utilize such gradients to influence delivery of a polynucleotide to a cell. DNA can be compacted by adding polycations to the mixture. By interacting an appropriate cation with a DNA containing system, DNA condensation can take place. Since the ion utilized for compaction may exist in higher concentration outside of the cell membrane compared to inside the cell membrane, this natural ionic gradient can be utilized in delivery systems.

Polymers

A polymer is a molecule built up by repetitive bonding together of smaller units called monomers. In this application the term polymer includes both oligomers which have two to about 80 monomers and polymers having more than 80 monomers. The polymer can be linear, branched network, star, comb, or ladder types of polymer. The polymer can be a homopolymer in which a single monomer is used or can be copolymer in which two or more monomers are used. Types of copolymers include alternating, random, block and graft.

To those skilled in the art of polymerization, there are several categories of polymerization processes that can be utilized in the described process. The polymerization can be chain or step. This classification description is more often used that the previous terminology of addition and condensation polymer.

Step Polymerization: In step polymerization, the polymerization occurs in a stepwise fashion. Polymer growth occurs by reaction between monomers, oligomers and polymers. No initiator is needed since there is the same reaction throughout and there is no termination step so that the end groups are still reactive. The polymerization rate decreases as the functional groups are consumed.

Typically, step polymerization is done either of two different ways. One way, the monomer has both reactive functional groups (A and B) in the same molecule so that

A-B yields−[A-B]

Or the other approach is to have two difunctional monomers.

A-A+B-B yields−[A-A-B-B]

Generally, these reactions can involve acylation or alkylation. Acylation is defined as the introduction of an acyl group (—COR) onto a molecule. Alkylation is defined as the introduction of an alkyl group onto a molecule.

“If functional group A is an amine then B can be (but not restricted to) an isothiocyanate, isocyanate, acyl azide, N-hydroxysuccinimide, sulfonyl chloride, aldehyde (including formaldehyde and glutaraldehyde), ketone, epoxide, carbonate, imidoester, carboxylate activated with a carbodiimide, alkylphosphate, arylhalides (difluoro-dinitrobenzene), anhydride, or acid halide, p-nitrophenyl ester, o-nitrophenyl ester, pentachlorophenyl ester, pentafluorophenyl ester, carbonylimidazole, carbonyl pyridinium, or carbonyl dimethylaminopyridinium. In other terms when function A is an amine then function B can be acylating or alkylating agent or amination agent.

If functional group A is a sulfhydryl then function B can be (but not restricted to) an iodoacetyl derivative, maleimide, aziridine derivative, acryloyl derivative, fluorobenzene derivatives, or disulfide derivative (such as a pyridyl disulfide or 5-thio-2-nitrobenzoic acid {TNB} derivatives).

If functional group A is carboxylate then function B can be (but not restricted to) adiazoacetate or an amine in which a carbodiimide is used. Other additives may be utilized such as carbonyldiimidazole, dimethylamino pyridine (DMAP), N-hydroxysuccinimide or alcohol using carbodiimide and DMAP.

If functional group A is an hydroxyl then function B can be (but not restricted to) an epoxide, oxirane, or an amine in which carbonyldiimidazole or N,N′-disuccinimidyl carbonate, or N-hydroxysuccinimidyl chloroformate or other chloroformates are used. If functional group A is an aldehyde or ketone then function B can be (but not restricted to) an hydrazine, hydrazide derivative, amine (to form a Schiff Base that may or may not be reduced by reducing agents such as NaCNBH3) or hydroxyl compound to form a ketal or acetal.

Yet another approach is to have one bifunctional monomer so that A-A plus another agent yields -[A-A]-. If function A is a sulfhydryl group then it can be converted to disulfide bonds by oxidizing agents such as iodine (I2) or NaIO4 (sodium periodate), or oxygen (O2). Function A can also be an amine that is converted to a sulfhydryl group by reaction with 2-Iminothiolate (Traut's reagent) which then undergoes oxidation and disulfide formation. Disulfide derivatives (such as a pyridyl disulfide or 5-thio-2-nitrobenzoic acid{TNB} derivatives) can also be used to catalyze disulfide bond formation. Functional group A or B in any of the above examples could also be a photoreactive group such as aryl azide (including halogenated aryl azide), diazo, benzophenone, alkyne or diazirine derivative.

Reactions of the amine, hydroxyl, sulfhydryl, carboxylate groups yield chemical bonds that are described as amide, amidine, disulfide, ethers, esters, enamine, imine, urea, isothiourea, isourea, sulfonamide, carbamate, alkylamine bond (secondary amine), carbon-nitrogen single bonds in which the carbon contains a hydroxyl group, thioether, diol, hydrazone, diazo, or sulfone”.

If functional group A is an aldehyde or ketone then function B can be (but not restricted to) an hydrazine, hydrazide derivative, amine (to form a Schiff Base that may or may not be reduced by reducing agents such as NaCNBH3) or hydroxyl compound to form a ketal or acetal.

Yet another approach is to have one difunctional monomer so that

A-A plus another agent yields -[A-A]-.

If function A is a sulfhydryl group then it can be converted to disulfide bonds by oxidizing agents such as iodine (I2) or NaIO4 (sodium periodate), or oxygen (O2). Function A can also be an amine that is converted to a sulfhydryl group by reaction with 2-iminothiolate (Traut's reagent) which then undergoes oxidation and disulfide formation. Disulfide derivatives (such as a pyridyl disulfide or 5-thio-2-nitrobenzoic acid{TNB} derivatives) can also be used to catalyze disulfide bond formation.

Functional group A or B in any of the above examples could also be a photoreactive group such as aryl azides, halogenated aryl azides, diazo, benzophenones, alkynes or diazirine derivatives.

Reactions of the amine, hydroxyl, sulfhydryl, carboxylate groups yield chemical bonds that are described as amide, amidine, disulfide, ethers, esters, enamine, urea, isothiourea, isourea, sulfonamide, carbamate, carbon-nitrogen double bond (imine), alkylamine bond (secondary amine), carbon-nitrogen single bonds in which the carbon contains a hydroxyl group, thio-ether, diol, hydrazone, diazo, or sulfone.

Chain Polymerization: In chain-reaction polymerization growth of the polymer occurs by successive addition of monomer units to limited number of growing chains. The initiation and propagation mechanisms are different and there is usually a chain-terminating step. The polymerization rate remains constant until the monomer is depleted.

Monomers containing vinyl, acrylate, methacrylate, acrylamide, methaacrylamide groups can undergo chain reaction which can be radical, anionic, or cationic. Chain polymerization can also be accomplished by cycle or ring opening polymerization. Several different types of free radical initiatiors could be used that include peroxides, hydroxy peroxides, and azo compounds such as 2,2′-Azobis(-amidinopropane) dihydrochloride (AAP). A compound is a material made up of two or more elements.

Types of Monomers: A wide variety of monomers can be used in the polymerization processes. These include positive charged organic monomers such as amines, imidine, guanidine, imine, hydroxylamine, hydrozyine, heterocycles (like imidazole, pyridine, morpholine, pyrimidine, or pyrene. The amines could be pH-sensitive in that the pKa of the amine is within the physiologic range of 4 to 8. Specific amines include spermine, spermidine, N,N′-bis(2-aminoethyl)-1,3-propanediamine (AEPD), and 3,3′-Diamino-N,N-dimethyldipropylammonium bromide. Monomers can also be hydrophobic, hydrophilic or amphipathic.

Amphipathic compounds have both hydrophilic (water-soluble) and hydrophobic (water-insoluble) parts. Hydrophilic groups indicate in qualitative terms that the chemical moiety is water-preferring. Typically, such chemical groups are water soluble, and are hydrogen bond donors or acceptors with water. Examples of hydrophilic groups include compounds with the following chemical moieties carbohydrates; polyoxyethylene, peptides, oligonucleotides and groups containing amines, amides, alkoxy amides, carboxylic acids, sulfurs, or hydroxyls. Hydrophobic groups indicate in qualitative terms that the chemical moiety is water-avoiding. Typically, such chemical groups are not water soluble, and tend not to hydrogen bond. Hydrocarbons are hydrophobic groups. Monomers can also be intercalating agents such as acridine, thiazole organge, or ethidium bromide.

Lipids are amphipathic compounds which are a fat. Fat is a glyceryl ester of fatty acids. Fatty acids is a term that is used to describe the group of substances which are soluble in hydrocarbons and insoluble in water. They may be saturated or unsaturated.

Other Components of the Monomers and Polymers: The polymers have other groups that increase their utility. These groups can be incorporated into monomers prior to polymer formation or attached to the polymer after its formation. These groups include: Targeting Groups—such groups are used for targeting the polymer-nucleic acid complexes to specific cells or tissues. Examples of such targeting agents include agents that target to the asialoglycoprotein receptor by using asiologlycoproteins or galactose residues. Other proteins such as insulin, EGF, or transferrin can be used for targeting. Protein refers to a molecule made up of 2 or more amino acid residues connected one to another as in a polypeptide. The amino acids may be naturally occurring or synthetic. Peptides that include the RGD sequence can be used to target many cells. Peptide refers to a linear series of amino acid residues connected to one another by peptide bonds between the alpha-amino group and carboxyl group of contiguous amino acid residues. Chemical groups that react with sulfhydryl or disulfide groups on cells can also be used to target many types of cells. Folate and other vitamins can also be used for targeting. Other targeting groups include molecules that interact with membranes such as fatty acids, cholesterol, dansyl compounds, and amphotericin derivatives.

After interaction of the supramolecular complexes with the cell, other targeting groups can be used to increase the delivery of the drug or nucleic acid to certain parts of the cell. For example, agents can be used to disrupt endosomes and a nuclear localizing signal (NLS) can be used to target the nucleus.

A variety of ligands have been used to target drugs and genes to cells and to specific cellular receptors. The ligand may seek a target within the cell membrane, on the cell membrane or near a cell. Binding of ligands to receptors typically initiates endocytosis. Ligands could also be used for DNA delivery that bind to receptors that are not endocytosed. For example peptides containing RGD peptide sequence that bind integrin receptor could be used. In addition viral proteins could be used to bind the complex to cells. Lipids and steroids could be used to directly insert a complex into cellular membranes.

The polymers can also contain cleavable groups within themselves. When attached to the targeting group, cleavage leads to reduce interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines.

Reporter or marker molecules are compounds that can be easily detected. Typically they are fluorescent compounds such as fluorescein, rhodamine, texas red, CY-5, CY-3 or dansyl compounds. They can be molecules that can be detected by UV or visible spectroscopy or by antibody interactions or by electron spin resonance. Biotin is another reporter molecule that can be detected by labeled avidin. Biotin could also be used to attach targeting groups.

A polycation is a polymer containing a net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polymer containing a net negative charge, for example polyglutamic acid. The polyanion can contain monomer units that are charge negative, charge neutral, or charge positive, however, the net charge on the polymer must be negative. A polyanion can also mean a non-polymeric molecule that contains two or more negative charges. The term polyion includes polycation, polyanion, zwitterionic polymers, and neutral polymers. The term zwitterionic refers to the product (salt) of the reaction between an acidic group and a basic group that are part of the same molecule. Salts are ionic compounds that dissociate into cations and anions when dissolved in solution. Salts increase the ionic strength of a solution, and consequently decrease interactions between nucleic acids with other cations. A charged polymer is a polymer that contains residues, monomers, groups, or parts with a positive or negative charge and whose net charge can be neutral, positive, or negative.

Signals

In a preferred embodiment, a chemical reaction can be used to attach a signal to a nucleic acid complex. The signal is defined in this specification as a molecule that modifies the nucleic acid complex and can direct it to a cell location (such as tissue cells) or location in a cell (such as the nucleus) either in culture or in a whole organism. By modifying the cellular or tissue location of the foreign gene, the expression of the foreign gene can be enhanced.

The signal can be a protein, peptide, lipid, steroid, sugar, carbohydrate, nucleic acid or synthetic compound. The signals enhance cellular binding to receptors, cytoplasmic transport to the nucleus and nuclear entry or release from endosomes or other intracellular vesicles.

Nuclear localizing signals enhance the targeting of the gene into proximity of the nucleus and/or its entry into the nucleus. Such nuclear transport signals can be a protein or a peptide such as the SV40 large T ag NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus.

Signals that enhance release from intracellular compartments (releasing signals) can cause DNA release from intracellular compartments such as endosomes (early and late), lysosomes, phagosomes, vesicle, endoplasmic reticulum, Golgi apparatus, trans Golgi network (TGN), and sarcoplasmic reticulum. Release includes movement out of an intracellular compartment into cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides.

Cellular receptor signals are any signal that enhances the association of the gene or particle with a cell. This can be accomplished by either increasing the binding of the gene to the cell surface and/or its association with an intracellular compartment, for example: ligands that enhance endocytosis by enhancing binding the cell surface. This includes agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with sulfhydryl or disulfide groups on cells can also be used to target many types of cells. Folate and other vitamins can also be used for targeting. Other targeting groups include molecules that interact with membranes such as lipids fatty acids, cholesterol, dansyl compounds, and amphotericin derivatives. In addition viral proteins could be used to bind cells.

The present invention provides compounds used in systems for the transfer of polynucleotides, oligonucleotides, and other compounds into association with cells within tissues in situ and in vivo.

The process of delivering a polynucleotide to a cell has been commonly termed “transfection” or the process of “transfecting” and also it has been termed “transformation”. The polynucleotide could be used to produce a change in a cell that can be therapeutic. The delivery of polynucleotides or genetic material for therapeutic and research purposes is commonly called “gene therapy”. The polynucleotides or genetic material being delivered are generally mixed with transfection reagents prior to delivery.

A biologically active compound is a compound having the potential to react with biological components. More particularly, biologically active compounds utilized in this specification are designed to change the natural processes associated with a living cell. For purposes of this specification, a cellular natural process is a process that is associated with a cell before delivery of a biologically active compound. In this specification, the cellular production of, or inhibition of a material, such as a protein, caused by a human assisting a molecule to an in vivo cell is an example of a delivered biologically active compound. Pharmaceuticals, proteins, peptides, polypeptides, hormones, cytokines, antigens, viruses, oligonucleotides, and nucleic acids are examples of biologically active compounds.

Polynucleotides

The term polynucleotide, or nucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxyaminomethyl-2-thiouracil, β-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs encoding siRNA or antisense genes, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a gene(s). The term recombinant as used herein refers to a polynucleotide molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more proteins. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The polynucleotide may contain sequences that do not serve a specific function in the target cell but are used in the generation of the polynucleotide. Such sequences include, but are not limited to, sequences required for replication or selection of the polynucleotide in a host organism.

The terms naked nucleic acid and naked polynucleotide indicate that the nucleic acid or polynucleotide is not associated with a transfection reagent or other delivery vehicle that is required for the nucleic acid or polynucleotide to be delivered to the cell. A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

Vectors are polynucleic molecules originating from a virus, a plasmid, or the cell of a higher organism into which another nucleic fragment of appropriate size can be integrated without loss of the vectors capacity for self-replication; vectors typically introduce foreign DNA into host cells, where it can be reproduced. Examples are plasmids, cosmids, and yeast artificial chromosomes; vectors are often recombinant molecules containing DNA sequences from several sources. A vector includes a viral vector: for example, adenovirus; DNA; adenoassociated viral vectors (AAV) which are derived from adenoassociated viruses and are smaller than adenoviruses; and retrovirus (any virus in the family Retroviridae that has RNA as its nucleic acid and uses the enzyme reverse transcriptase to copy its genome into the DNA of the host cell's chromosome; examples include VSV G and retroviruses that contain components of lentivirus including HIV type viruses).

A non-viral vector is defined as a vector that is not assembled within an eukaryotic cell.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by hybridization, multistrand polynucleotide formation, homologous recombination, gene conversion, or other yet to be described mechanisms.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the term gene expression refers to the process of converting genetic information encoded in a gene into RNA (e.g., mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

Ionic (electrostatic) interactions are the non-covalent association of two or more substances due to attractive forces between positive and negative charges, or partial positive and partial negative charges.

Condensed Nucleic Acids: Condensing a polymer means decreasing the volume that the polymer occupies. An example of condensing nucleic acid is the condensation of DNA that occurs in cells. The DNA from a human cell is approximately one meter in length but is condensed to fit in a cell nucleus that has a diameter of approximately 10 μm. The cells condense (or compacts) DNA by a series of packaging mechanisms involving the histones and other chromosomal proteins to form nucleosomes and chromatin. The DNA within these structures is rendered partially resistant to nuclease DNase) action. The process of condensing polymers can be used for delivering them into cells of an organism.

Condensed nucleic acids may be delivered intravasculary, intrarterially, intravenously, orally, intraduodenaly, via the jejunum (or ileum or colon), rectally, transdermally, subcutaneously, intramuscularly, intraperitoneally, intraparenterally, via direct injections into tissues such as the liver, lung, heart, muscle, spleen, pancreas, brain (including intraventricular), spinal cord, ganglion, lymph nodes, lymphatic system, adipose tissues, thyroid tissue, adrenal glands, kidneys, prostate, blood cells, bone marrow cells, cancer cells, tumors, eye retina, via the bile duct, or via mucosal membranes such as in the mouth, nose, throat, vagina or rectum or into ducts of the salivary or other exocrine glands. “Delivered” means that the polynucleotide becomes associated with the cell. The polynucleotide can be on the membrane of the cell or inside the cytoplasm, nucleus, or other organelle of the cell.

An intravascular route of administration enables a polymer or polynucleotide to be delivered to cells more evenly distributed and more efficiently expressed than direct injections. Intravascular herein means within a tubular structure called a vessel that is connected to a tissue or organ within the body. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein.

An administration route involving the mucosal membranes is meant to include nasal, bronchial, inhalation into the lungs, or via the eyes.

Recharging Condensed Nucleic Acids

Polyions for gene therapy and gene therapy research can involve anionic systems as well as charge neutral or charge-positive systems. The ionic polymer can be utilized in “recharging” (another layer having a different charge) the condensed polynucleotide complex. The resulting recharged complex can be formed with an appropriate amount of charge such that the resulting complex has a net negative, positive or neutral charge. The interaction between the polycation and the polyanion can be ionic, can involve the ionic interaction of the two polymer layers with shared cations, or can be crosslinked between cationic and anionic sites with a crosslinking system (including cleavable crosslinking systems, such as those containing disulfide bonds). The interaction between the charges located on the two polymer layers can be influenced with the use of added ions to the system. With the appropriate choice of ion, the layers can be made to disassociate from one another as the ion diffuses from the complex into the cell in which the concentration of the ion is low (use of an ion gradient).

Electrostatic complexes between water-soluble polyelectrolytes have been studied widely in recenty ears. Complexes containing DNA as a polyanionic constituent only recently came to the attention because of their potential use in gene therapy applications such as non-viral gene transfer preparations (polyplexes) for particle delivery to a cell. Strong polyelectrolytes, polyanion/polycation complexes, are usually formed at a 1:1 charge stoichiometrically. A charge ratio 1:1 complex between DNA and Poly-L-Lysine (PLL) also has been demonstrated in the prior art.

Polyanions effectively enhance the gene delivery/gene expression capabilities of all major classes of polycation gene delivery reagents. In that regard, we disclose the formation of negatively charged tertiary complexes containing nucleic acid, PLL, and succinic anhydride-PLL (SPLL) complexes. SPLL is added to a cationic nucleic acid/PLL complex in solution. Nucleic acid at the core of such complexes remains condensed, in the form of particles ˜50 nm in diameter. DNA and PLL binds SPLL in 1:1:1 complex with SPLL providing a net negative charge to the entire complex. Such small negatively charged particles are useful for non-viral gene transfer applications.

One of the advantages that flow from recharging DNA particles is reducing their non-specific interactions with cells and serum proteins [(Wolfert et al. Hum. Gene Therapy 7:2123-2133 (1996); Dash et al., Gene Therapy 6:643-650 (1999); Plank et al., Hum. Gene Ther. 7:1437-1446 (1996); Ogris et al., Gene Therapy 6:595-605 (1999); Schacht et al. Brit. Patent Application 9623051.1 (1996)]

A wide a variety of polyanions can be used to recharge the DNA/polycation particles. They include (but not restricted to): Any water-soluble polyanion can be used for recharging purposes including succinylated PLL, succinylated PEI (branched), polyglutamic acid, polyaspartic acid, polyacrylic acid, polymetbacrylic acid, polyethylacrylic acid, polypropylacrylic acid, polybutylacrylic acid, polymaleic acid, dextran sulfate, heparin, hyaluronic acid, polysulfates, polysulfonates, polyvinyl phosphoric acid, polyvinyl phosphonic acid, copolymers of polymaleic acid, polyhydroxybutyric acid, acidic polycarbohydrates, DNA, RNA, negatively charged proteins, pegylated derivatives of above polyanions, pegylated derivatives carrying specific ligands, block and graft copolymers of polyanions and any hydrophilic polymers (PEG, poly(vinylpyrrolidone), poly(acrylamide), etc).

These polyanions can be added prior to the nucleic acid complex being delivered to the cell or organism. In one preferred embodiment the recharged nucleic acid complexes (polyanion/polycation/nucleic acid complex) are formed in a container and then administered to the cell or organism. In another preferred embodiment, the polycation/nucleic acid complex is recharged with a polyion prior to delivery to the organism and the nucleic acid remains condensed. In this embodiment the nucleic acid can remain more than 50%, 60%, 70%, 80%, 90% or 100% condensed as well.

When an excess of polyion is present, DNA forms soluble condensed (toroid) structures stabilized with an excess of polyion. When, in addition to this binary complex, a third polyelectrolyte is present, a tertiary complex exists. In the absence of salt such tertiary complex might exist indefinitely. If the last added polyion is in excess, it stabilizes the complex in the form of a soluble colloid. Using this method, a DNA/polycation complex, which maintains a net positive charge, reverses its charge and becomes “recharged”. The complex can be designed (e.g. choice of polycation and polyanion, presence of crosslinking) so that in the presence of salt, the complex dissociates into binary complex and free excess of third polyion.

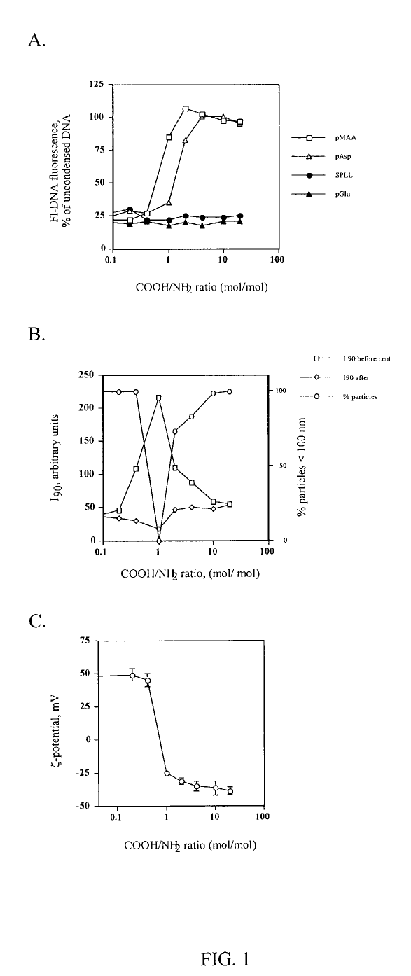

In general, tertiary DNA/PLL/SPLL complex exhibit the same colloid properties as binary DNA/PLL complex. In low salt solution it forms flocculate around PLL/SPLL charge equivalence point (FIG. 1B).

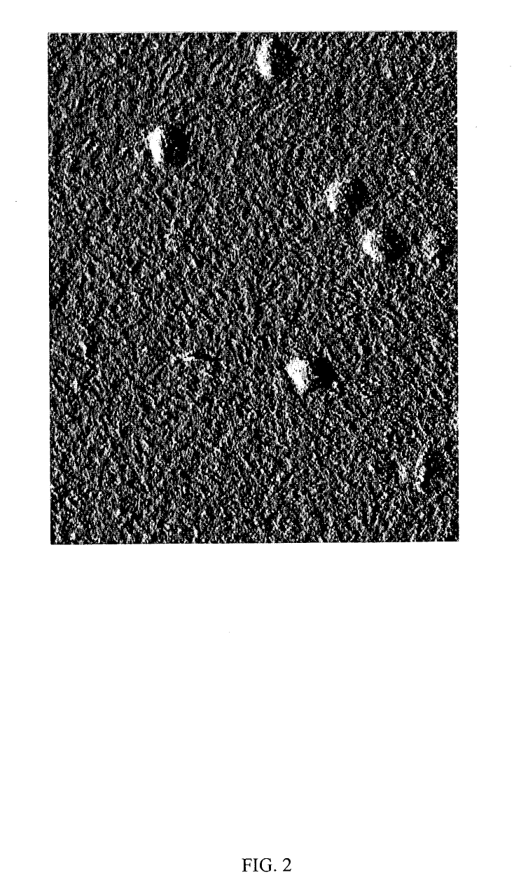

DNA condensation assays based on the effect of concentration-dependent self-quenching of covalently-bound fluorophores upon DNA collapse indicated essentially the same phenomenon described in the prior art. Polyanions with high charge density (polymethacrylic acid, PMAA and polyaspartic acid, pAsp) were able to decondense DNA complexed with PLL while polyanions with lower charge density (polyglutamic acid, pGlu, SPLL) failed to decondense DNA (FIG. 1A). Together with z-potential measurements (FIG. 1C), these data represent support for the presence of negatively charged condensed DNA particles. These particles are approximately 50 nm in diameter in low salt buffer as measured by atomic force microscopy (FIG. 2) which revealed particles of spheroid morphology.

The issue of stoichiometry in such tertiary complexes is of primary importance to determine how much polyanion is associated with DNA after formation of tertiary complex and potential dissociation of polycation after polyanion binding. We developed a methodology for DNA complex stoichiometry determination which includes step density gradient ultracentrifugation of complexes prepared with fluorescently labeled DNA, PLL and SPLL. Retrieved complexes were always found aggregated and possess DNA/PLL/SPLL (1:1:1) stoichiometry. This surprising finding assumes major redistribution of charges inside the particle since net charge of the complex is negative. Excess PLL was found to complex with any excess SPLL.

In another preferred embodiment, the polyanion can be covalently attached to the polycation using a variety of chemical reactions without the use of crosslinker. The polyanion can contain reactive groups that covalently attach to groups on the polycation. The types of reactions are similar to those discussed above in the section on step polymerization.

In another preferred embodiment the attachment of the recharged complex can be enhanced by using chelators and crown ethers, preferably polymeric.

Excess of the polycations or polyanions can be toxic or interfere with nucleic acid delivery and transfection. In one preferred embodiment the DNA/polycation complexes are initially formed by adding only a small excess of polycation to nucleic acid (in charge ratio which is defined as ratio of polycation total charge to polyanion total charge at given pH). The charge ratio of polycation to nucleic acid charge could be less than 2, less than 1.7, less than 1.5 or even less than 1.3. This would be preferably done in low ionic strength solution so as to avoid the complexes from flocculation. Low ionic strength solution means solution with total monovalent salt concentration less than 50 mM. Then the polyanion is added to the mixture and only a small amount of “blank” particles are formed. “Blank” particles are particles that contain only polycation and polyanion and no nucleic acid.

In another preferred embodiment, the polycation is added to the nucleic acid in charge excess but the excess polycation that is not in complex with the nuclei acid is removed by purificaton. Purification means removing of charged polymer using centrifugation, dialysis, chromatography, electrophoresis, precipitation, extraction.

Yet in another preferred embodiment a ultracentrifugation procedure (termed “centrifugation step”) is used to reduce the amount of excess polycation, polyanion, or “blank” particles. The method is based on the phenomenon that only dense DNA-containing particles can be centrifuged through 10% sucrose solution at 25,000 g. After centrifugation purified complex is at the bottom of the tube while excess of polyanion and “blank” particles stay on top. In modification of this experiment 40% solution of metrizamide can be used as a cushion to collect purified DNA/polycation/polyanion complex on the boundary for easy retrieval.

The attachment of the polyanion to the DNA/polycation complex enhance stability but can also enable a ligand or signal to be attached to the DNA particle. This is accomplished by attaching the ligand or signal to the polyanion which in turn is attached to the DNA particle. A dialysis step or centifugation step can be used to reduce the amount of free polyanion containing a ligand or signal that is in solution and not complexed with the DNA particle. One approach is to replace the free, uncomplexed polyanion containing a ligand or signal with free polyanion that does not contain a ligand or signal.

Yet in another preferred embodiment a polyanion used for charge reversal is modified with neutral hydrophilic polymer for steric stabilization of the whole complex. The complex formation of DNA with pegylated polycations results in substantial stabilization of the complexes towards salt- and serum-induced flocculation (Wolfert et al. Hum. Gene Therapy 7:2123-2133 (1996), Ogris et al., Gene Therapy 6:595-605 (1999). We have demonstrated that modification of polyanion in triple complex also significantly enhances salt and serum stability.

In another preferred embodiment a polyanion used for charge reversal is cleavable. One can imagine two ways to design a cleavable polyion: 1. A polyion cleavable in backbone, 2. A polyion cleavable in side chain. First scenario would comprise monomers linked by labile bonds such as disulfide, diols, diazo, ester, sulfone, acetal, ketal, enol ether, enol ester, imine and enamine bonds. Second scenario would involve reactive groups (i.e. electrophiles and nucleophiles) in close proximity so that reaction between them is rapid. Examples include having corboxylic acid derivatives (acids, esters and amides) and alcohols, thiols, carboxylic acids or amines in the same molecule reacting together to make esters, thiol esters, anhydrides or amides. In one specific preferred embodiment the polyion contains an ester acid such as citraconnic acid, or dimethylmaleyl acid that is connected to a carboxylic, alcohol, or amine group on the polyion.

Cleavable means that a chemical bond between atoms is broken. Labile also means that a chemical bond between atoms is breakable. Crosslinking refers to the chemical attachment of two or more molecules with a bifunctional reagent. A bifunctional reagent is a molecule with two reactive ends. The reactive ends can be identical as in a homobifunctional molecule, or different as in a heterobifucnctional molecule.

Angiogenesis

The term, angiogenesis, in this specification is defined as any formation of new blood vessels. Angiogenesis may also refer to the sprouting of new blood vessels (endothelium-lined channels such as capillaries) from pre-existing vessels as a result of proliferation and migration of endothelial cells. The maturation or enlargement of vessels via recruitment of smooth muscle cells, i.e. the formation of collateral arteries from pre-existing arterioles, is termed arteriogenesis. Vasculogenesis refers to the in situ formation of blood vessels from angioblasts and endothelial precursor cells (EPCs). An anastomosis is a connection between two blood vessels. The formation of anastomoses can be important for restoring blood flow to ischemic tissue. The formation of new vessels in ischemic tissue or in other tissue with insufficient blood perfusion is termed revascularization. As used herein, the term angiogenesis encompasses arteriogenesis, vasculogenesis, anastomosis formation, and revascularization.

Angiogenesis is regulated by soluble secreted factors, cell surface receptors and transcription factors. Secreted factors include cytokines, chemokines, and growth factors that affect endothelial cells, smooth muscle cells, monocytes, leukocytes, and precursor cells. Such factors include: vascular endothelial growth factors, fibroblast growth factors, hepatocyte growth factors, angiopoietin 1 (Ang-1), angiopoietin 2 (Ang-2), Platelet derived growth factors (PDFGs), granulocyte macrophage-colony stimulating factor, insulin-like growth factor-1 (IGF-1), IGF-2, early growth response factor-1 (EGR-1), and human tissue kallikrein (HK).

Delivery of genes that encode angiogenic factors to cells in vivo provides an attractive alternative to repetitive injections of protein for the treatment of vascular insufficiency or occlusions. Genes that encode angiogenic factors, including both natural and recombinant secreted factors, receptors, and transcription factors, can be targeted to cells in the affected area, thereby limiting deleterious effects associated with delivering angiogenic factors throughout the body. In particular, according to the described invention, genes for angiogenic factors can be delivered to muscle cells in vivo, including skeletal and cardiac muscle cells. Expression of the gene and secretion of the gene product then induces angiogenesis and improves collateral blood flow in the targeted tissue. The improved blood flow can both improve muscle tissue function and relieve pain associated with vascular diseases.

### EXAMPLES

### Example 1

Materials. Plasmid DNA (pCILuc) used for the condensation studies was provided by Bayou Biolabs, Harahan, La. Poly-L-lysine (PLL) (MW 34 kDa), poly-L-aspartic acid (PAA) (MW 36 kDa), poly-L-glutamic acid (PLG) (MW 49 kDa) and rhodamine B isothiocyanate were products of Sigma (St. Louis, Mo.). Polymethacrylic acid (PMA), metrizamide and fluoresceine isothiocyanate were from Aldrich (Milwaukee, Wis.). LabelIT kits (Mirus Corp., Madison, Wis.) were used for covalent labeling DNA with fluorescein and rhodamine.

Synthesis of succinylated PLL (SPLL). Succinic anhydride (30 mg) dissolved in 150 μl DMSO were added to PLL (20 mg) dissolved in 1 ml of 0.1 M sodium tertraborate solution in two portions. After 10 min incubation at RT, the polymer was precipitated with two volumes of isopropanol with subsequent reconstitution with deionized water.

Labeling of PLL and DNA with fluorescein and rhodamine. Fluorescein isothiocyanate (0.37 mg in 5 μl DMSO) was added to PLL (20 mg) in 1 ml of sodium tertraborate and incubated for 1 h. Resulting F1-PLL was purified by isopropanol precipitation. F1-PLL was used also for preparation of F1-SPLL by succinylation as described above. For DNA labeling, DNA and LabelIT reagent (Mirus Corp., Madison, Wis.) were mixed in HEPES buffer (25 mM HEPES, pH 7.5) in reagent/DNA weight ratios of 1:1 and incubated for 1 h at 37° C. Labeled DNA was precipitated two times with NaCl/ethanol mixture (final NaCl concentration was 0.2 M, ethanol 66%) and immediately redissolved in deionized water

DNA/polyion complex formation. DNA/PLL/SPLL complexes were formed in 25 mM HEPES, pH 7.5 at DNA concentration 20-100 μg/ml. The complex with DNA/PLL charge ratio (1:3) was formed by consecutive addition of PLL and then various amount of SPLL and vortexing for 30 sec.

Light scattering and zeta-potential measurements. Intensity of scattered light measured at 90° angle (I90) was estimated using Shimadzu RF 1501 set at ex=600 nm; em=600 nm. Particle sizing and zeta-potential measurements were performed using a Zeta Plus Particle Analyzer (Brookhaven Instruments Corp., Holtsville, N.Y.), with a laser wavelength of 532 nm.

Atomic force microscopy. Images of DNA particles were obtained using BioProbe AFM microscope (Park Scientific instruments, Sunnyvale, Calif.). Samples (DNA concentration 1 μg/ml in 25 mM HEPES, pH 7.5) were allowed to adsorb on mica in the presence of 1 mM NiCl₂ for 5 min and then were viewed in the buffer in a contact mode.

Ultracentrifugation experiments. For stoichiometry studies, tertiary complexes were formed using fluorescently labeled polyions. Two types of complexes were formed in 25 mM HEPES, pH 7.5, (charge ratio 1:3:10): a) Rh-DNA/F1-PLL/SPLL and b) Rh-DNA/PLL/F1-SPLL. The samples (1 ml) were layered on top of 10% sucrose solution (10 ml) with 1 ml of 40% metrizamide cushion on the bottom and were centrifuged in SW-41 Beckman rotor in Optima LE-80K ultracentrifuge at 30,000 rpm for 20 min. DNA-containing complexes were retrieved from sucrose/metrizamide boundary using Pasteur pipet and were dissolved in 2.5 M NaCl solution. Visible spectra of the complexes and 1:1 premixed Rh-DNA/F1-PLL and Rh-DNA/F1-SPLL standards (700-400 nm) were recorded using Shimadzu UV 1601 spectrophotometer.

### Example 2

Recharging of Polyion Condensed DNA Particles: The chief DNA/polycation complex used was DNA/PLL (1:3 charge ratio) formed in low salt buffer. At these conditions, plasmid DNA is completely condensed and compacted into toroid-shaped soluble particles stabilized with excess of polyion (Kabanov et al. Adv. Drug Delivery Rev 30:49-60 (1998). The DNA particles were characterized after addition of a third polyion component to such binary DNA/polyion complex. It has been shown that polyanion (polymer or negatively-charged lipid bilayer) can release DNA from its complex with cationic liposomes. As judged by DNA condensation assay based on ethidium bromide binding, upon addition of such polyanions as dextran sulfate or heparin to the DNA/DOTAP lipid complexes results in release of free DNA. Using a fluorescein-labeled DNA condensation assay (Trubetskoy et al. Anal. Biochem. 267:309-313(1999) we demonstrate that the same is true for DNA/synthetic polyion complexes (FIG. 1A).

The aggregation state of condensed DNA particles was determined using both static and dynamic light scattering techniques. Upon titration of DNA/PLL (1:3) complex with increasing amounts of SPLL in low salt solution, turbidity of the reaction mixture, an indication of aggregation, increases when the lysine to lysyl succinate (NH2/COOH) ratio approaches 1:1 (FIG. 1(B)). With an excess of polyanion, turbidity decreases. Correspondingly, assessment of particle size by dynamic light scattering shows that small DNA particles (<100 nm) exist before and after the equivalent point. Large aggregates are present only at a 1:1 charge ratio of polyion to polyanion.

FIG. 1C demonstrates the change of particle surface charge (zeta potential) during titration of DNA/PLL (1:3) particles with SPLL. The particle becomes negatively charged and accordingly recharged at approximately the equivalence point (FIG. 1C).

Thus, upon addition of large excess of non-decondensing polyanion small non-aggregated particles still exist, DNA is still condensed but the charge of the particles becomes negative. We used atomic force microscopy to visualize these negatively charged particles. FIG. 2 shows small and non-aggregated 50 nm DNA/PLL/SPLL spheroids adsorbed on mica in the presence of 1 mM NiCl₂.

Any water-soluble polyanion can be used for recharging purposes including succinylated PLL, succinylated PEI, polyglutamic acid, polyaspartic acid, polyacrylic acid, polymethacrylic acid, dextran sulfate, heparin, hyaluronic acid, DNA, RNA, negatively charged proteins, polyanions graft-copolymerized with hydrophilic polymer, and the same carrying specific ligands.

### Example 3

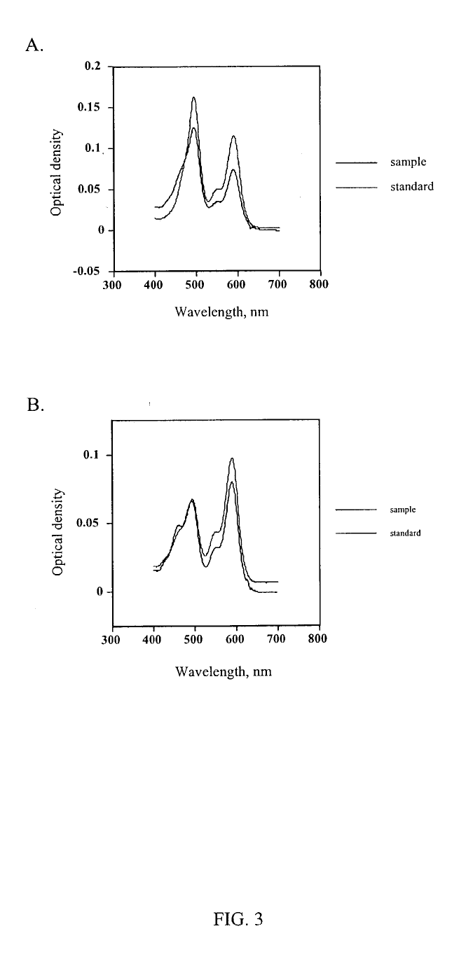

Stochiometry of Purified Particles: To study the stoichiometry of the recharged complexes, DNA, PLL and SPLL polymers were labeled with rhodamine and fluorescein moieties to yield Rh-DNA, F1-PLL and F1-SPLL with known degree of modification and adsorption coefficients respectively. Rh-DNA/F1-PLL/SPLL and Rh-DNA/PLL/F1-SPLL complexes were formed in low salt buffer and then separated from non-bound polyelectrolyte using density gradient ultracentrifugation. Corresponding amounts of each constituent can be determined by measuring optical density at 495 nm and 595 nm respectively. DNA complexes sediment through 10% sucrose solution and are retained in the separating layer between 10% sucrose and 40% metrizamide (metrizamide cushion). All Rh-DNA was found to be located on the sucrose/metrizamide border. Non-bound PLL and SPLL were found not to enter the 10% sucrose layer. DNA/PLL/SPLL complexes were found non-soluble and form precipitate on the density layer. The recovered complexes were solubilized in 2.5 M NaCl and their visible spectra were analyzed. FIG. 3 represents Rh-DNA/F1-PLL/SPLL (FIG. 3A) and Rh-DNA/PLL/F1-SPLL (FIG. 3B) complex spectra respectively together with standard Rh-DNA/F1-PLL and Rh-DNA/F1-SPLL (1:1) charge ratio mixtures. The data clearly indicates that precipitated complex contains all three polyelectrolytes with a stoichiometry of a 1:1:1 charge ratio.

### Example 4

Zeta Potential of Purified Particles: As one may conclude from stoichiometry studies, the DNA/PLL/SPLL (1:3:10) initial mixture along with 7× excess of free SPLL also contains 2× excess of PLL/SPLL particles (“blank particles”) not complexing DNA. These particles were found not to enter the 10% sucrose layer ensuring complete separation of DNA containing particles from PLL and SPLL excess. Zeta potential was measured using Brookhaven Instruments Corp. Zeta Plus Zeta Potential Analyzer. DNA concentration was 20 mg/ml in 1.5 ml of 25 mM HEPES, pH 7.5.

### Example 5

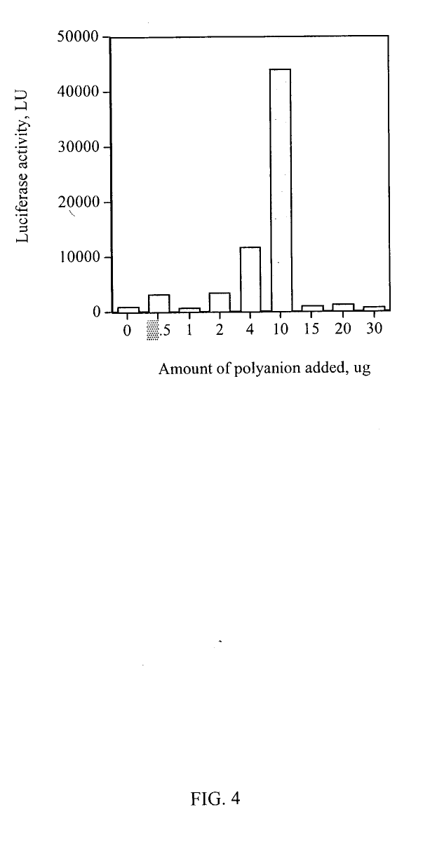

In vitro transfection enhancement upon recharging of DNA/polycation complexes. Recharging can increase the transfection activity of DNA/polycation complexes. FIG. 4 shows the results of transfection of HUH7 liver cells in 100% bovine serum with DNA/PEI (1:2 w/w) complexes recharged with increasing amounts of SPLL (MW=460 kDa). At optimal SPLL concentration activity of recharged complex exceeds the activity of the non-recharged one approximately 40 times. For transfection of recharged complexes, 2 μg of the reporter plasmid pCILuc (expressing the firefly luciferase cDNA from the human immediate early CMV promoter) (Zhang, G., Vargo, D., Budker, V., Armstrong, N., Knechtle, S. & Wolff, J. Human Gene Therapy 8, 1763-1772 (1997)) was complexed with the polycation and polyanion in low salt buffer. Resulting complexes were added to 35 mm wells containing cells at about 60% confluence. Transfected cells were harvested 48 h after transfection and cells were lysed and analyzed for luciferase activity using a Lumat LB 9507 luminometer (EG&G Berthold).

### Example 6

Recharged DNA/PEI complexes have reduced toxicity and exhibit gene transfer activity in vivo in an organism. Recharging of DNA/polycation complexes with strong polyanions which help to release DNA can also make complexes less toxic in vivo. Resulting complexes also are active in gene transfer in lungs upon i/v administration in mice. Table 1 shows the toxicity of DNA/PEI/dextran sulfate (DS) complex is decreasing with the increase of DS content. Tertiary DNA/PEI/dextran sulfate complexes were formed in 290 mM glucose, 5 mM HEPES, pH 7.4 at DNA concentration of 0.2 mg/ml and PEI concentration of 0.4 mg/ml. Each animal was injected 0.25 ml of DNA complex solution. After 24 h, the animals were sacrificed, lungs, livers, hearts, kidneys were removed and homogenized at 4° C. Luciferase activity of extracts (10 μl) was measured using a Lumat LB 9507 luminometer (EG&G Berthold).

TABLE 1
In vivo gene transfer activity in mouse organs upon i/v
administration of DNA/PEI/PAA complexes (50 μg/100 μg).

| | Luciferase Activity, LU | | | |
|---|---|---|---|---|
| | 40 μg PAA | 50 μg PAA | 60 μg PAA | 70 μg PAA |
| Liver | 1465 | 3266 | 14537 | 387 |
| Lung | 182187 | 9392 | 325 | 162335 |
| Spleen | 3752 | 1925 | 1647 | 1307 |
| Heart | 2186 | 158 | 76 | 1262 |
| animal | 1/3 | 1/4 | 0/3 | 0/3 |
| survival | | | | |
| (dead/total) | | | | |

### Example 7

Crosslinking of polycation and polyanion layers on the DNA-containing particles increases their stability in serum and on the cell surface.

Negatively charged (recharged) particles of condensed DNA can possess the same physico-chemical properties as positively charged (non-recharged) ones. This includes flocculation in high salt solutions (including physiologic concentration). We found that chemical cross-linking of cationic and anionic layers of the DNA particles can substantially improve stability of the particles in serum as well as on the cell surface. Table 2 shows the time course of unimodal particle size of DNA/PLL/SPLL crosslinked and non-crosslinked particles in 80% bovine serum as determined by dynamic light scattering.

TABLE 2
Particle sizing of DNA/PLL/SPLL crosslinked and non-
crosslinked complexes in 80% serum.

| Time, min | size (nm) no | |
|---|---|---|
| crosslinking | crosslinking | size (nm) |
| 0 | 153 | 104 |
| 15 | 154 | 105 |
| 60 | 171 | 108 |
| 200 | 246 | 115 |

Crosslinked particles essentially do not change their size in 200 min at RT while non-crosslinked control flocculates rapidly. Crosslinking with cleavable reagents might help to overcome an inactivity problem. The polymers can also contain cleavable groups within themselves. When attached to the targeting group, cleavage leads to reduce interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines, acyl hydrazones, and Schiff bases.

### Example 8

Pegylation of polyanions for recharging. Recharging of DNA/polycation particles with PEG-polyanion conjugates can substantially stabilize recharged particles against salt-induced flocculation. Preparation of PEG-SPLL conjugate. Water-soluble carbodiimide (EDC, 5 mg,) and N-hydroxysulfosuccinimide (S-NHS, 10 mg) were added to the 0.25 ml solution of SPLL (20 mg/ml, Mw=210 kDa) at pH 5.0 and incubated for 5 min at RT. Monoamino-polyethleneglycol (4 mg, 0.4 ml in 0.1 M HEPES, pH 8.0) was added to the SPLL and the mixture was contained to incubate for 1 more hour. PEG-SPLL conjugate was dialysed against deionized water overnight at 4° C. and freeze-dried. This preparation resulted in 5% (mol) substitution of COOH groups with PEG chains.

DNA-containing particles were prepared using the procedure in Example 1 with the exception that SPLL-PEG conjugate was doubled compared to SPLL. Table 3 shows the time course of unimodal particle size of DNA/PLL/SPLL and DNA/PLL/PEG-SPLL particles in 80% bovine serum as determined by dynamic light scattering. Pegylated particles exhibit higher stability towards flocculation as opposed to non-pegylated ones.

TABLE 3
Particle sizing of DNA/PLL/polyanion complexes recharged
with SPLL and PEG-SPLL in 80% serum.

| Time, min | Size (nm) SPLL | Size (nm) PEG-SPLL |
|---|---|---|
| 0 | 441 | 118 |
| 15 | 750 | 118 |
| 60 | 2466 | 139 |
| 120 | 5494 | 116 |

### Example 9

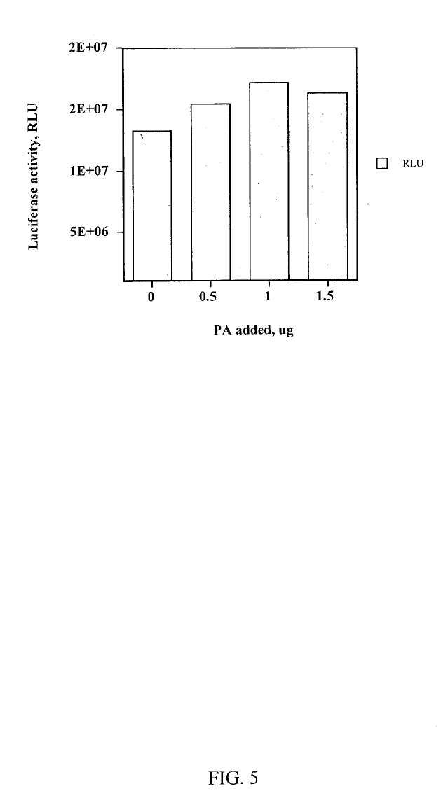

Enhancement of in vitro transgene activity of DNA/lipofectamine (Gibco/BRL, 3:1 DNA/lipid ratio) complexes with PA treatment. The complexes were formed in OPTI-MEM culture medium at DNA concentration 50 μg/ml. Polyglutamic acid was added after 5 min incubation. Subconfluently seeded 293 cells in 6-well were treated with 2 μg DNA (pCILuc plasmid) for 3 h following addition of full medium. After 48 h the cells were scraped and assayed for gene expression (luciferase). The data if FIG. 5 show that transfection activity of DNA/lipid complexes is enhanced by recharging.

### Example 10

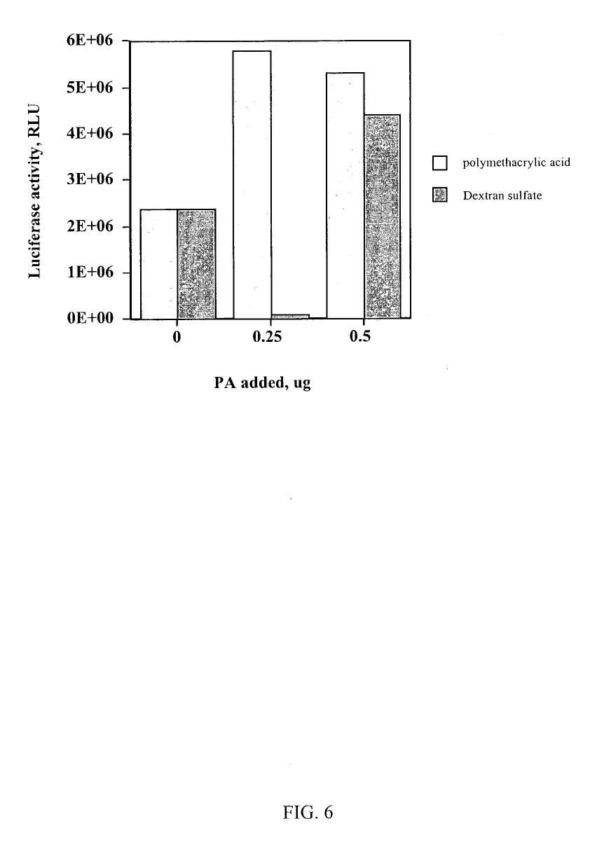

Enhancement of in vitro transgene activity of DNA/LT1 (Mirus Corp., 3:2 DNA/lipid ratio, w/w) complexes with PA treatment. Complexes were formed as specified in Example 1. Results, shown in FIG. 6, demonstrate that recharging of DNA-containing complexes enhances in vitro action activity.

### Example 11

Preparation of DNA/DOTAP:cholesterol complexes recharged with polyacrylic acid (PAA). DOTAP and cholesterol were mixed in 2:1 molar ratio and dispersed in 5% glucose solution buffered with 5 mM HEPES (IG solution) and briefly sonicated in a bath-type lab sonicator. Luciferase-encoding plasmid pCILuc (50 μg) was complexed with CL containing 530 μg of DOTAP and 150 μg of cholesterol in 250 μL of IG solution. Different amounts of PAA (10-60 μg) were added to each preparation.

### Example 12

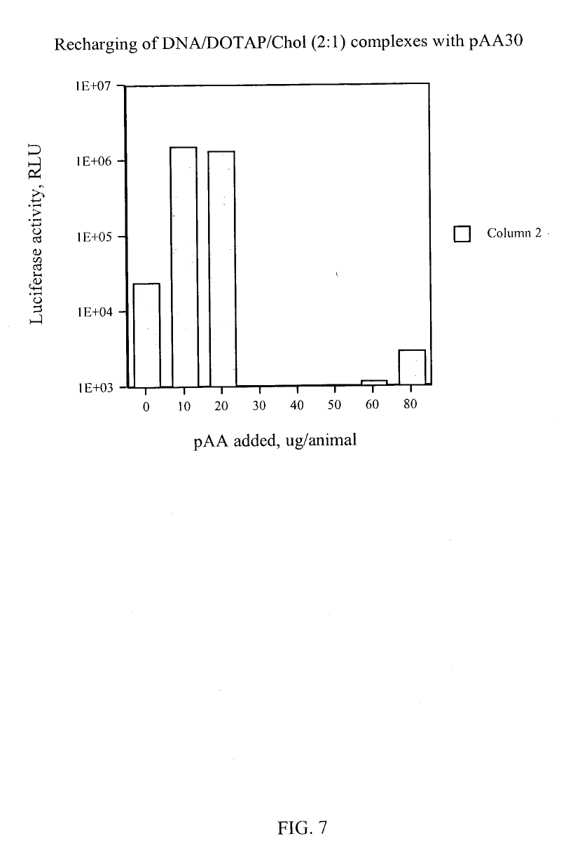

Use of DNA/DOTAP:cholesterol complexes recharged with PAA for enhancement of gene delivery in lung. Mice were injected via tail vein with 250 μl of PAA recharged complexes (50 μg DNA/animal). Lungs were harvested and homogenized at 4° C. after 24 h. Luciferase activity of extracts (10 μL) was measured using a Lumat LB 9507 luminometer (EG&G Berthold). FIG. 7 shows the enhancement of transgene activity in lungs upon addition of PAA. Complete flocculation of the sample occurred in the range of 30-50 μg of PAA added. The data demonstrates almost two orders of magnitude increase on transgene activity in lungs after recharging DNA/CL complexes with strong polyanion and essentially no activity past flocculation point.

### Example 13

Hepatocytes delivery of cross-linked tertiary DNA/PLL/SPLL complexes by tail vein injection.

Materials:

Plasmid DNA (pCILuc) were labeled with Cy3 LablelT(Mirus Corporation, Madison Wis.). Labeled DNA were typically dissolved in water at concentrations ranged from 1.5-2 mg/ml. Poly-L-Lysine, PLL (MW 31 kDa), dissolved in water at 10 mg/ml was purchased from Sigma Chemicals (St. Louis, Mo.). Succinylated PLL (SPLL) was prepared as previously described and dissolved in water at 20 mg/ml.

DNA/PLL/SPLL cross-linked tertiary complexes were formed at a charge ratio of 1:3:10 as follows for a single injection:

SPLL (345 μg in 50 μl of 20 mM MES, pH 5) were activated with the addition of 292 μg of EDC followed by 583 μg sulfo-NHS, both were dissolved in H20 at 100 mg/1.2 ml, and incubated for 10 min. At the end of the activation period, 50 μg of cy3-labeled DNA in 100 μl of 20 mM MES, pH 6.5 was added to 95 μg of PLL in 100 ul of 20 mM MES, pH 6.5 and mixed immediately. The condensed DNA/PLL complexes were added immediately to the activated SPLL solution and mixed thoroughly. The cross-linked particles were allowed to incubate at RT for at least 2 h before in-vivo injections. Typically, majority of the particles size ranged from 60-200 nm with an average size around 130 nm and a Zeta-potential of −40 mV. Salt and serum stability of particles were evaluated by particles size changes over time in the presence of physiologic salt solution or serum.

The cross-linked particles solution containing 50 μg of Cy3-DNA in 250 μl were injected into a mouse through the tail vein. After 3 h, the animal was sacrificed, liver samples were submerged in HistoPrep (Fisher Scientific) and snapped frozen in liquid nitrogen. Frozen liver sections, 4-5 μm thick, were prepared and were counter stained sequentially for 20 min each by 10 nm Sytox green (Molecular Probe) in PBS for cell nuclei and 15 ng/ml of Alexa 488 phalloidin (Molecular Probe) in PBS for actin filaments. Stained slides were analyzed for hepatocytes uptake of Cy3-DNA containing particles using a Zeiss laser scanning confocal microscope.

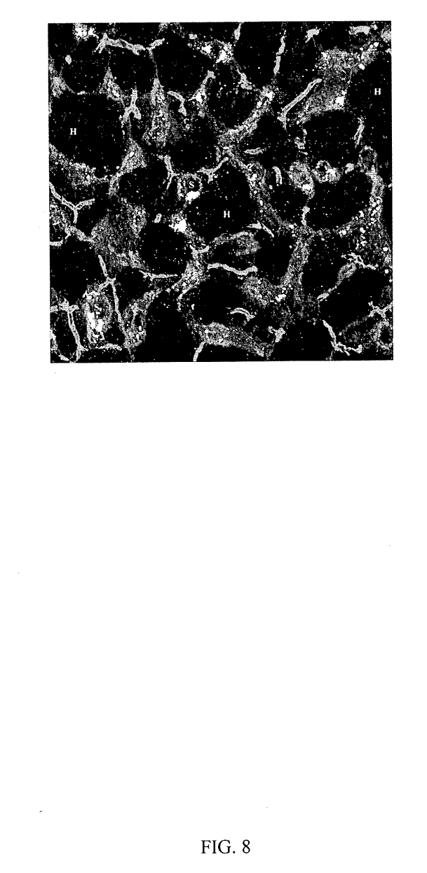

FIG. 8 shows the fluorescence signals from 10 consecutive confocal planes superimposed to form one image, each plane was 0.45 μm thick. With the average size of a mouse hepatocyte around 25-30 μm thick, the composite image roughly represent ¼ of total signals per hepatocytes. It showed that each cell contained 20-40 punctate signals. Each punctate signal may represent endosomes at various stages of the pathway and may contain one or more DNA containing particles. Hepatocytes were distinguishable by their larger size in comparison to other cells and bi-nucleated for a large percentage of the population. A few of the hepatocytes were indicated by (H). A large number of particles were also found in Kuppfer and endothelial cells. These sinusoidal cells were smaller in size, possessed very little cytoplasm space and were indicted by (S). Red=DNA containing tertiary complex. Green=cell nuclei and actin filaments which were localized primarily along the cell surface and with the strongest signal along bile canaliculi.

### Example 14

Hepatocytes delivery of DNA/polyallylamine-cysteine/polyacrylic acid-thioester complexes

Materials:

Synthesis of polyallylamine-cysteine (pAllylamine-cys) conjugate: N,N′-bis(t-BOC)-L-cystine (37 mg, 0.08 mmol) was dissolved in 5 mL methylene chloride to this was added N-hydroxysuccinimide (21 mg, 2.2 eq) and dicyclohexylcarbodiimide (37 mg, 2.2 eq). The solution was allowed to stir overnight at RT. The dicyclohexylurea was removed by filtering the solution through a cotton plug in a Pasteur pipette. The succinimidyl ester was then added, with rapid stirring, to a solution of polyallylamine hydrochloride MW 50,000 (10 mg, 0.8 eq) that had been dissolved in a solution of methanol (20 mL) and diisopropylethylamine (0.5 mL). After one hour, the solvents were removed by rotary evaporation. The white solid was then dissolved in trifluoroacetic acid (5 mL), triisopropylsilane (0.25 mL), and water (0.25 mL). After 2 h, the solvents were removed by rotary evaporation. The resulting solid was then dissolved in water (25 mL) and the pH was adjusted to 9 by the addition of potassium carbonate. To this solution was added β-mercaptoethanol (1 mL). After 2 h, the pH was adjusted to 2 by the addition of hydrochloride and the solution was placed into dialysis tubing (MWC 12,000) and dialyzed against 2 L of water that was adjusted to pH 2 with addition of hydrochloric acid. The dialysis solution was changed four times over 48 h. After dialysis the solution contained 1.3 mg/mL polyallylamine, which is 14 mM of amine functional groups. Analysis of the thiol content of the solution by reaction with 5,5′-dithiobis(2-nitrobenzoic acid) in pH 7.5 100 mM phosphate buffer and quantification by comparison to solutions containing a known amount of β-mercaptoethanol revealed 2.7 mM of thiol functional groups, an 18% modification of all functional groups.

Synthesis of polyacrylic acid thioester (pAA-thioester): To a solution of mercaptoacetic acid (1 mL) in 10 mL methylene chloride was added polyacryloyl chloride MW 10,000 (100 mg). After 30 min, the methylene chloride was removed by rotary evaporation and the resulting oil was dissolved in 20 mL water and dialyzed against 2 L water. The dialysis solution was changed four times over a 72 h period. The amount of thioester was quantified by measuring the absorbance of the thioester at 230 nm using the extinction coefficient of 3,800 M⁻¹ cm⁻¹ (Anal. Biochem. 1985, 150, 121) and was determined to be at 80% modification of all functional groups.

Complexes for injection were formulated in 250 μl of 5 mM HEPES buffer, pH 8. For a single injection, 20 μg of pAllylamine-cys was added to 10 μg of Cy3-DNA. Polyacrylic acid thioester (60 ug) was then added to the condensed complex and let incubate overnight at 4° C. Amide bonds were formed as interactions occurred between the cysteine groups and the thioester groups. These cross-linked particles had an average diameter of 94 nm in size and a Zeta-potential of −40 mV. Particle stability were evaulated by changes of particles size in the presence of physiologic salt and serum. Injection of complexes and analysis for hepatocyte delivery were essentially the same as described in example 1.

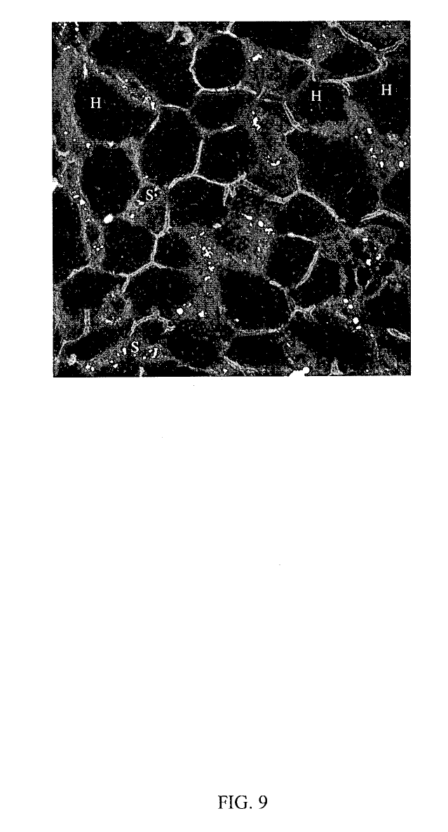

FIG. 9 shows the delivery of Cy3-DNA/pAllyamine-cys/pAA-thioester particles, 1 to 5 particles per hepatocytes, to at least 60% of the hepatocytes. Considering the lower concentration of DNA injected, the efficiency of hepatocytes delivery was comparable to that of Cy3-DNA/PLL/SPLL complexes. Similar to Cy3-DNA/PLL/SPLL complexes, sinusoidal cells (mostly endothelial and Kupffer cells) also contained a large number of particles. Black=DNA containing complexes. Grey=cell nuclei and actin filaments. This example represent another method of cross-linking to formulate liver targetable negatively charged particles.

### Example 15

### Increased Vascularization Following Delivery of a Therapeutic Polynucleotide to Primate Limb

DNA delivery was performed via brachial artery with blood flow blocked by a sphygmomanometer cuff proximately to the injection site. Left arm was transfected with VEGF, while right arm was transfected with EPO. The Sartorious musle from left leg was used as non-injected control. A male Rhesus monkey weighing 14 kg was used for these injections. The animal was anesthetized with Ketamin (10-15 mg/kg). A modified pediatric blood pressure cuff was positioned on the upper arm. The brachial artery was cannulated with a 4 F angiography catheter. The catheter was advanced so that the tip was positioned just below the blood pressure cuff. Prior to the injection, the blood pressure cuff was inflated so that the cuff pressure was at least 20 mmHg higher than the systolic blood pressure. After cuff inflation, papaverine (5 mg in 30 ml of saline) was injected by hand (˜8 to 10 seconds). After 5 min, the pDNA solution was delivered rapidly with a high volume injection system. For the EPO injection, 10 mg of pDNA was added to 170 ml of saline and injected at a rate of 6.8 ml per second. For the VEGF injection, 10 mg of pDNA was added to 150 ml of saline, and injected at a rate of 5.4 ml per second.

After 65 days, the animal was euthanized by overdose I.V. injection of pentobarbital Ketamin (10 mg/kg). The entire Pronator quadratus and Pronator teres MUSCLES from both sides were immediately harvested and fixed for 3 day in 10% neutral buffered formalin (VWR, Cleveland, Ohio). After fixation, an identical grossing was performed for left and right muscles and slices across the longitudinal muscles were taken. Specimens were routinely processed and embedded into paraffin (Sherwood Medical, St. Louis, Mo.). Four microns sections were mounted onto precleaned slides, and stained with hematoxylin and eosin (Surgipath, Richmond, Ill.) for pathological evaluation. Sections were examined under Axioplan-2 microscope and pictures were taken with the aid of AxioCam digital camera (both from Carl Zeiss, Goettingen, Germany).

To evaluate the effect of VEGF plasmid delivery on cell composition in muscle tissue and neo-angiogenesis, we used monoclonal mouse anti-human CD31 antibody (DAKO Corporation, Carpinteria Calif.). The immunostaining was performed using a standard protocol for paraffin sections. Briefly: four microns paraffin sections were deparaffinized and re-hydrated. Antigen retrieval was performed with DAKO Target Retrieval Solution (DAKO Corporation, Carpinteria Calif.) for 20 min at 97° C. To reduce non-specific binding the section were incubated in PBS containing 1% (wt/vol) BSA for 20 min at RT. Primary antibody 1:30 in PBS/BSA were applied for 30 min at RT. CD31 antibody were visualized with donkey anti-mouse Cy3-conjugated IgG, 1:400 (Jackson Immunoresearch Lab, West Grove Pa.) for 1 h at RT. ToPro-3 (Molecular Probes Inc.) was used for nuclei staining; 1:70,000 dilution incubated for 15 min at RT. Sections were mounted with Vectashield non-fluorescent mounting medium and examined under confocal Zeiss LSM 510 microscope (Carl Zeiss, Goettingen, Germany). Images were collected randomly under 400× magnification, each image representing 0.106 sq mm. Because muscle fibers and red blood cells have an autofluorescence in FITC channel we use 488 nm laser to visualize these structures. Morphometry analysis. Coded mages were opened in Adobe Photoshop 5.5 having image size 7×7 inches in 1×7 inches window, and a grid with rulers was overlaid. The number of muscle fibers, CD31 positive cells and total nuclei was counted in all 7 image's strips consecutively, without any knowledge of experimental design. T-Test for Two-Sample Unequal Variances was used for statistical analysis.

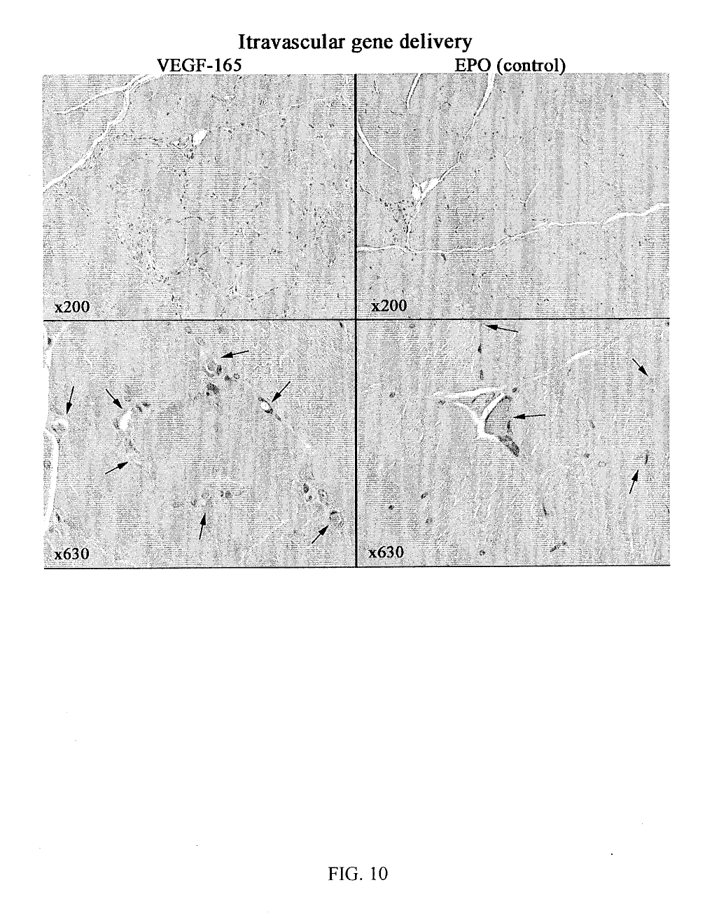

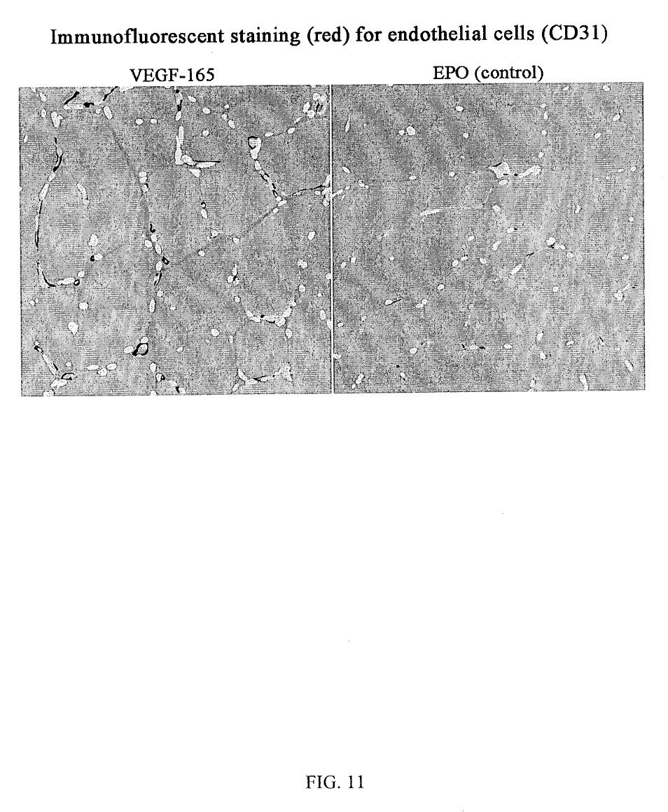

Results: Microscopic evaluation did not reveal any notable pathology in either muscle regardless of the gene delivered. Also, neither muscle showed any notable presence of inflammatory cells, except of few macrophages. Necrosis of single muscle fibers was extremely rare in both, occupying negligible volume and was not associated with infiltration/vascularization. However, in muscles transfected with VEGF-165 plasmid, the interstitial cell and vascular density (observed in H&E-stained slides) was obviously increased (FIG. 10), as compare to EPO plasmid administered muscle (FIG. 10). Based on morphologic evaluation, these newly arrived interstitial cells we suggested to be endothelial and adventitial cells, smooth muscle cells, and fibroblasts. To evaluate participation of endothelial cells in this neo-morphogenesis, we have counted the number of CD31 positive cells in EPO and VEGF delivered Pronator quadratus MUSCLES (FIG. 11). To assure that comparable specimens were analyzed in right and left muscles, the number of muscle fibers was counted per area unit (0.106 sq mm). The VEGF and EPO administered muscles were not different in muscle fiber number (means 30.5 and 31.6). The number of CD31 positive cells however was significantly increased by 61.7% p<0.001 (means 53.2 vs 32.9).

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

We claim:

1. A process delivering a protein or peptide to a muscle tissue of a patient for improving blood flow in the tissue comprising:
a) forming a compound having a net charge comprising a polynucleotide encoding the peptide or protein and a polymer in a solution;
b) adding a charged polymer to the solution in sufficient amount to form a complex having a net charge different from the compound net charge;
c) injecting the complex into a blood vessel lumen, in vivo;
d) increasing permeability in the blood vessel; and,
e) delivering the complex to an extravascular muscle cell outside of the blood vessel via the increased permeability, wherein the polynucleotide is expressed.

2. The process of claim 1 wherein improving blood flow consists of stimulating new blood vessel formation.

3. The process of claim 1 wherein the peptide or protein consists of an angiogenic factor.

4. The process of claim 3 wherein the angiogenic factor consists of vascular endothelial growth factor.

5. The process of claim 4 wherein the vascular endothelial growth factor is selected from the list consisting of: VEGF, VEGF II, VEGF-B, VEGF-C, VEGF-D, VEGF-E, VEGF₁₂₁, VEGF₁₃₈, VEGF₁₄₅, VEGF₁₆₅, VEGF₁₈₉ and VEGF₂₀₆.

6. The process of claim 3 wherein the angiogenic factor consists of fibroblast growth factor.

7. The process of claim 6 wherein the fibroblast growth factor is selected from the list consisting of: FGF-1, FGF-1b, FGF-1c, FGF-2, FGF-2b, FGF-2c, FGF-3, FGF-3b, FGF-3c, FGF-4, FGF-5, FGF-7, FGF-9, acidic FGF and basic FGF.

8. The process of claim 1 wherein the blood vessel consists of a coronary vessel.

9. The process of claim 1 wherein the blood vessel consists of a limb artery.

10. The process of claim 1 wherein the limb artery consists of the femoral artery.

11. The process of claim 1 wherein the permeability of the vessel is increased by inserting papaverine into the vessel prior to or together with the polynucleotides.

12. The process of claim 1, wherein delivery of the polynucleotide stimulates angiogenesis in the muscle tissue.

13. The process of claim 1 wherein enhancing blood flow consists of improving collateral blood flow.

14. The process of claim 13 wherein improving collateral blood flow consists of stimulating collateral blood vessel formation.

15. The process of claim 1 wherein the muscle tissue is affected by a vascular occlusion.

16. The process of claim 1 wherein the muscle tissue is not affected by a vascular occlusion.

17. The process of claim 1 wherein the muscle tissue is suffering from ischemia.

18. The process of claim 1 wherein the muscle tissue is not suffering from ischemia.

19. The process of claim 1 wherein the muscle tissue is heart muscle tissue.

20. The process of claim 19 wherein the heart muscle tissue is human heart muscle tissue.

21. The process of claim 19 wherein delivery of the polynucleotide improves abnormal cardiac function.

22. The process of claim 1 wherein the muscle tissue is skeletal muscle tissue.

23. The process of claim 22 wherein the skeletal muscle tissue is limb skeletal muscle tissue.

24. The process of claim 23 wherein the limb skeletal muscle tissue is human limb skeletal muscle tissue.

25. The process of claim 1 wherein the patient has peripheral vascular disease.

26. The process of claim 1 wherein the patient has peripheral arterial occlusive disease.

27. The process of claim 1 wherein the patient has peripheral-deficient vascular disease.

28. The process of claim 1 wherein the patient has myocardial ischemia.

29. The process of claim 26 wherein the patient suffers from claudication or intermittent claudication.

30. The process of claim 26 wherein delivery of the polynucleotide results in decreased pain associated with a peripheral circulatory disorder.

31. The process of claim 1 wherein the peptide or protein is secreted from the muscle cell.

32. The process of claim 1 wherein the peptide or protein stimulates vascular cell growth.

33. The process of claim 1 wherein delivery of the polynucleotide stimulates vascular cell migration.

34. The process of claim 1 wherein delivery of the polynucleotide stimulates vascular cell proliferation.

35. A process delivering polynucleotides to a muscle tissue for improving blood flow in the tissue comprising:
a) forming a compound having a net charge comprising a polynucleotide and a polymer in a solution;
b) adding a charged polymer to the solution in sufficient amount to form a complex having a net charge different from the compound net charge;
c) injecting the complex into a blood vessel lumen, in vivo; and,
d) delivering the complex to an extravascular muscle cell outside of the blood vessel via the increased permeability.

36. The process of claim 35 wherein the polynucleotide consists of an RNA function inhibitor.

37. The process of claim 36 wherein the RNA function inhibitor consists of siRNA.

38. The process of claim 37 wherein the siRNA blocks expression of an angiogenesis inhibitor.
